(19) United States
(12) Patent Application Publication
(10) Pub. No.: US 2019/0127787 A1
(43) Pub. Date: May 2, 2019

(54) METHOD OF MULTI-GENE ABSOLUTE QUANTIFICATION FOR PCR ARRAY

(71) Applicant: Quark Biosciences, Inc., Hsinchu County (TW)

(72) Inventors: Ming-Chou Lee, Hsinchu County (TW); You-Xiu Zheng, Hsinchu County (TW)

(73) Assignee: Quark Biosciences, Inc., Hsinchu County (TW)

(21) Appl. No.: 16/177,462

(22) Filed: Nov. 1, 2018

Related U.S. Application Data
(60) Provisional application No. 62/580,986, filed on Nov. 2, 2017.

(51) Int. Cl. C12Q 1/6851

(52) U.S. Cl. CPC C12Q 1/6851

(57) ABSTRACT

A method of multi-gene absolute quantification for PCR array is provided, wherein a nucleic acid sample to be tested includes at least one kind of nucleic acid target. Primers for amplifying a plurality of standard DNA or the nucleic acid target to be tested are respectively disposed in a plurality of reaction wells of a test carrier. Afterwards, the plurality of standard DNA with known copy number and the nucleic acid sample are mixed and added into the reaction wells, and qPCR is performed on the standard DNA and the nucleic acid sample. A sequence-specific combination of a forward primer and a reverse primer is designed for each standard DNA, and the same DNA template sequence is presented between regions corresponding to the forward primer and the reverse primer. The primers for amplifying standard DNA and the nucleic acid target to be tested have similar amplification efficiencies.

### CROSS-REFERENCE TO RELATED APPLICATIONS

This application claims the priority benefit of U.S. provisional Patent Application No. 62/580,986, filed on Nov. 2, 2017. The entirety of the above-mentioned patent application is hereby incorporated by reference herein and made a part of this specification.

### BACKGROUND OF THE INVENTION

### Field of the Invention

The invention relates to a method of absolute quantification, and more particularly, to a method of multi-gene absolute quantification for PCR array.

### Description of Related Art

With the development of modern biomedical technology and clinical needs, it is necessary to perform absolute quantification of target genes in many diagnostic methods. For example, it is necessary to perform absolute quantification of target genes in the viral load and treatment monitoring of infections such as human immunodeficiency virus (HIV) or hepatitis C virus (HCV), or viral load monitoring of organ transplant patients. In the prior art, it is necessary to dispose the experimental standards and the sample to be tested in different reaction wells if a 96-well plate is used in qPCR technique to perform the test, so as to prevent interference. Therefore, a relatively large number of reaction wells is required, which is counterproductive to the experimental process and the testing cost.

Based on the above, for a test carrier having a plurality of reaction wells, how to facilitate the experiment and reduce the testing cost when performing absolute quantification on nucleic acid samples via qPCR technique is an important topic.

### SUMMARY OF THE INVENTION

The invention provides a method of multi-gene absolute quantification for PCR array. The method of the invention is able to facilitate the experiment and reduce the testing cost effectively when performing absolute quantification on nucleic acid samples via qPCR technique, especially for a test carrier having a plurality of reaction wells.

The method of multi-gene absolute quantification for PCR array of the invention includes the following steps, wherein a nucleic acid sample to be tested includes at least one kind of nucleic acid target. Primers for amplifying a plurality of standard DNA or a nucleic acid target to be tested are respectively disposed in a plurality of reaction wells of a test carrier. Next, the plurality of standard DNA with known but different copy numbers and the nucleic acid sample to be tested are mixed and added into a plurality of reaction wells, and qPCR is performed on the plurality of standard DNA and the nucleic acid sample. Each standard DNA has a sequence-specific combination of a forward primer and a reverse primer designed for each of the standard DNA, so as to prevent interference between the plurality of standards and interference between the standards and the sample to be tested. As a result, the plurality of standard DNA can be mixed and independently amplified in the same reaction without affecting each other. Moreover, the primers for amplifying the plurality of standard DNA and the nucleic acid target to be tested have similar amplification efficiencies, and the plurality of standard DNA have the same DNA template sequence between regions corresponding to the forward primer and the reverse primer. Therefore, no difference exists during the amplification of the plurality of standard DNA and the nucleic acid target to be tested, so as to accurately determine the copy number of the test object.

In an embodiment of the invention, after a standard curve is created according to the logarithm of the copy number [log(copy number)] and the Cq values of the plurality of standard DNA, with the logarithm of the copy number [log(copy number)] as the horizontal axis and the Cq values as the vertical axis, the Cq value of the nucleic acid target is substituted into the standard curve to obtain the copy number of the nucleic acid target.

In an embodiment of the invention, the amplification efficiencies include a Tm value.

In an embodiment of the invention, the plurality of reaction wells of the test carrier are divided into a plurality of clusters, wherein each cluster is formed by the plurality of reaction wells, and the same kind of primers for amplifying the standard DNA or the nucleic acid target are disposed in the plurality of reaction wells of each cluster.

In an embodiment of the invention, the reaction wells of each cluster are in a 3×3 format.

In an embodiment of the invention, each of the clusters is used to perform qPCR on one of the plurality of standard DNA or a single kind of the nucleic acid target.

Based on the above, the invention provides a method of multi-gene absolute quantification for PCR array, wherein the plurality of standard DNA have different forward primers and reverse primers respectively, and a DNA template sequence with the same amplification efficiency is presented between the forward primers and the reverse primers. As a result, the efficiency deviation between the plurality of standard DNA can be prevented. Moreover, the primers for amplifying the plurality of standard DNA and the nucleic acid target to be tested also have similar amplification efficiencies. Therefore, the plurality of standard DNA and a nucleic acid sample are mixed and added into reaction wells in the invention, such that a standard curve can be created according to the copy number and the Cq values of the plurality of standard DNA, so as to extrapolate the copy number of the nucleic acid target. In addition, since each forward primer and reverse primer has a different DNA sequence, interference between the plurality of standard DNA and interference between the standard DNA and the sample to be tested can be effectively prevented, and it is not necessary to dispose the standard

DNA and the sample to be tested in different reaction wells for testing. As a result, the drawback of having to use additional wells to perform testing on standards in the prior art can be alleviated, such that the experimental process is facilitated and the testing cost is reduced.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a standard curve created according to the logarithm of the copy number [log(copy number)] and the Cq values of 5 standard DNA of the invention.

### DESCRIPTION OF THE EMBODIMENTS

The invention provides a method of multi-gene absolute quantification for PCR array. In the following, the terms used in the specification are defined first.

“qPCR” or “real-time quantitative PCR” (real-time quantitative polymerase chain reaction) refers to an experimental method of using PCR to amplify and quantify target DNA at the same time. Quantification is performed using a plurality of measuring chemical substances (including SYBR® green fluorescent dye or Taqman fluorescent report oligonucleotide probe, for instance), and real-time quantification is performed with the amplified DNA accumulated in the reaction after every amplification cycle.

“Cq value” is the number of amplification cycles when fluorescence intensity starts to be significantly increased in a qPCR operation process.

“Sample” refers to the nucleic acid sample being tested. For example, the sample may be a nucleic acid fragment (DNA or RNA) extracted from blood, tissue, or saliva. “Template” refers to a DNA, RNA, or miRNA strand having a specific sequence, and is also referred to as a biomarker or a nucleic acid target, and can be detected via a qPCR reaction.

“Test carrier having a plurality of reaction wells” refers to a slide plate having a plurality of reaction wells, wherein each reaction well is used to perform a qPCR reaction.

The invention provides a method of multi-gene absolute quantification for PCR array, wherein the nucleic acid sample includes at least one kind of nucleic acid target to be tested. The method includes the following steps. A test carrier having a plurality of reaction wells is provided first, and preferably the plurality of reaction wells of the test carrier are divided into a plurality of clusters for example, wherein each cluster is formed by a plurality of reaction wells (such as 3×3 reaction wells), but the invention is not limited thereto. Afterwards, primers for amplifying the plurality of standard DNA or the nucleic acid target to be tested are respectively disposed in the plurality of reaction wells of the test carrier. Preferably, the same kind of forward and reverse primers for amplifying the standard DNA or the nucleic acid target are disposed in the plurality of reaction wells of each cluster, such that qPCR is performed on one of the plurality of standard DNA or a single kind of nucleic acid target in each cluster, for instance. Next, a plurality of standard DNA with known but different copy numbers and the nucleic acid sample are mixed and added into the plurality of reaction wells, and qPCR is performed on the plurality of standard DNA and the nucleic acid sample. After that, a standard curve is created according to the logarithm of the copy number [log(copy number)] and the Cq values of the plurality of standard DNA, with the logarithm of the copy number [log(copy number)] as the horizontal axis and the Cq value as the vertical axis (as mentioned previously, the copy number of the plurality of standard DNA is known before the experiment). Lastly, the Cq value of the nucleic acid target is substituted into the standard curve, so as to obtain the copy number of the nucleic acid target.

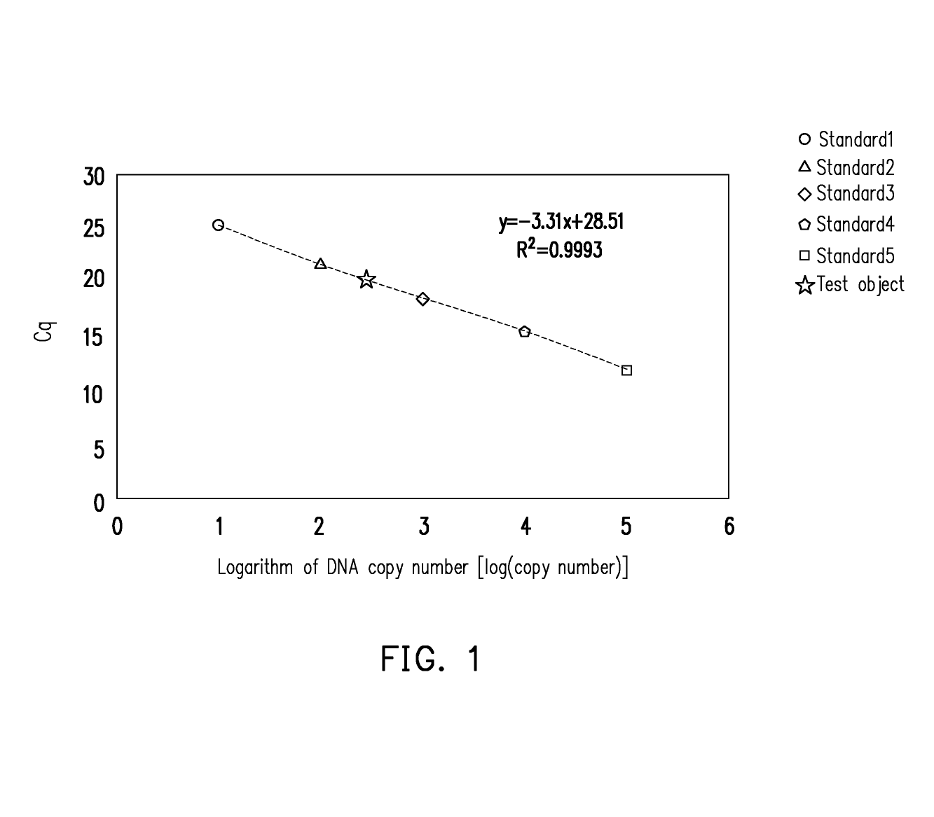

FIG. 1 is a standard curve created according to the logarithm of the copy number [log(copy number)] and the Cq values of 5 standard DNA of the invention. As shown in FIG. 1, 5 kinds of standard DNA are designed in the invention, and qPCR is performed on each standard DNA at a different concentrations. After that, a standard curve is created according to the logarithm of the copy number [log(copy number)] and the Cq values of the 5 standard DNA of the invention, with the logarithm of the DNA copy number [log(copy number)] as the horizontal axis and the Cq value as the vertical axis. The concentration of the test object can be obtained by the calculation of the Cq value thereof and the standard curve.

It should be mentioned that, in the present embodiment, the standards have different forward primers and reverse primers respectively, and the DNA sequences of the forward primers and the reverse primers of standard DNA are not the same. The primers with sequence specificity are able to prevent interference between the plurality of standard DNA and interference between the standard DNA and the sample to be tested, and it is not necessary to dispose the standard DNA and the sample to be tested in different reaction wells for testing. As for the number of standard DNA, five standard DNA may be used for example, but the invention is not limited thereto, and the number of standard DNA may be adjusted according to actual operation conditions, so as to draw a standard curve that meets experimental requirements. Moreover, the primers for amplifying the plurality of standard DNA and the nucleic acid target to be tested have similar amplification efficiencies, and the plurality of standard DNA have the same DNA template sequence between regions corresponding to the forward primer and the reverse primer. Therefore, no difference exists during the amplification of the plurality of standard DNA and the nucleic acid target to be tested, so as to determine the copy number of the test object accurately. In the present embodiment, the length of the forward primer and the reverse primer is 18 bp to 24 bp respectively, for example, and the length of the same DNA template sequence is 80 bp, for example.

In the following, the aforementioned absolute quantification method of the nucleic acid sample is described in detail via an experimental example. However, the following experimental example is not intended to limit the invention.

### EXPERIMENTAL EXAMPLE

### Reaction Condition Test

A reaction condition test was performed using 5 standard DNA and primer sets thereof to obtain optimal reaction conditions. qPCR was performed via a SYBR® green fluorescent dye with a test carrier having a plurality of reaction wells, and the resulting optimal reaction conditions are as follows, wherein about 90% to 100% PCR efficiency can be reached:

Primer concentration: 0.5 μm

Reaction temperature and time: 95° C. for 44 seconds, 60° C. for 88 seconds

Number of reaction cycles: 40 cycles

Quantification of standard DNA

The five standard DNA stock solutions (10 ng/μl) were diluted in series, then qPCR and digital PCR (dPCR) were respectively performed using a SYBR° green fluorescent dye with a test carrier having a plurality of reaction wells (125 wells/cluster, four repetitions per cluster), so as to extrapolate the concentrations of 5 standard DNA (copy number/μl). The standard curve and R² obtained from the serial dilution of the 5 standard DNA and the DNA concentrations (copy number/μl) measured by dPCR are listed in Table 1 below. Table 1 shows that the R² of the 5 standard DNA are all close to 1, indicating good serial dilution.

TABLE 1

| | | | DNA concentration |
|---|---|---|---|
| | Linear regression | | measured by dPCR |
| | equation | R² | (copy number/μl) |
| Standard DNA1 | y = 325387x − 0.0027 | 0.9970 | 2.17 × 10⁸ |
| Standard DNA2 | y = 283479x − 0.0078 | 0.9961 | 1.89 × 10⁸ |
| Standard DNA3 | y = 348829x − 0.0011 | 0.9999 | 2.33 × 10⁸ |
| Standard DNA4 | y = 397037x + 0.0578 | 0.9928 | 2.65 × 10⁸ |
| Standard DNA5 | y = 394724x − 0.0005 | 0.9982 | 2.63 × 10⁸ |

The actual concentrations of the standard DNA stock solutions extrapolated from the dPCR results and the Cq values obtained from the standard DNA with different concentrations were used to plot a standard curve with an equation of y=−3.3188x+25.672, wherein R² is 0.9987. The log(copy number/well) of the 5 standard DNA and the Cq values are listed in Table 2 below, and the PCR efficiency is 100.13%.

TABLE 2

| | log(copy number/well) | Cq |
|---|---|---|
| Standard DNA1 | 4.637 | 10.09 |
| Standard DNA2 | 3.577 | 13.90 |
| Standard DNA3 | 2.668 | 17.08 |
| Standard DNA4 | 1.724 | 19.91 |
| Standard DNA5 | 0.721 | 23.15 |

### Creating Calibration Curve Using Standard DNA

A standard curve was created using the actual concentrations of the standard DNA stock solutions extrapolated from the dPCR results and the Cq values obtained from 4 standard DNA with different concentrations, and then the concentration of the 5th standard DNA was calculated. The linear regression equation (y: Cq; x: log(copy number/well)), R², PCR efficiency, Cq value of target DNA and copy number/well extrapolated therefrom, and copy number/well detected by dPCR are listed in Table 3 below. As shown in Table 3, in the extrapolation results of the 5 standard DNA, the ratios of the copy number/well (A) calculated by the Cq values and the copy number/well (B) detected by dPCR are all close to 1. Therefore, it may be known that the results of the copy number/well (A) calculated by the Cq values and the copy number/well (B) detected by dPCR are quite close.

TABLE 3

| | | | | Target | Copy number/ | Copy number/ | |
|---|---|---|---|---|---|---|---|
| Linear regression | | PCR | Target | DNA Cq | well (A) calculated | well (B) detected | |
| equation | R² | efficiency | DNA | value | by Cq value | by dPCR | A/B |
| y = −3.2133x + 25.492 | 0.9992 | 104.47% | Standard | 10.09 | 6.21 × 10⁴ | 4.34 × 10⁴ | 1.43 |
| | | | DNA1 | | | | |
| y = −3.3319x + 25.68 | 0.9987 | 99.59% | Standard | 13.90 | 3.43 × 10³ | 3.78 × 10³ | 0.91 |
| | | | DNA2 | | | | |
| y = −3.3184x + 25.606 | 0.9995 | 100.15% | Standard | 17.08 | 3.71 × 10² | 4.66 × 10² | 0.80 |
| | | | DNA3 | | | | |
| y = −3.324x + 25.698 | 0.9986 | 99.91% | Standard | 19.91 | 5.51 × 10¹ | 5.30 × 10¹ | 1.04 |
| | | | DNA4 | | | | |
| y = −3.3858x + 25.917 | 0.9983 | 97.40% | Standard | 23.15 | 6.57 | 5.26 | 1.25 |
| | | | DNA5 | | | | |

After the average of the actual concentrations of the 5 standard DNA stock solutions extrapolated from the dPCR results was obtained, a standard curve was created using the Cq values obtained from the standard DNA with different concentrations. The equation is y=−3.213x+25.401, wherein R² is 0.9974. The log(average copy number/well) of the 5 standard DNA and the Cq values are listed in Table 4 below, and the PCR efficiency is 104.76%.

TABLE 4

| | log(average copy number/well) | Cq |
|---|---|---|
| Standard DNA1 | 4.669 | 10.09 |
| Standard DNA2 | 3.669 | 13.90 |
| Standard DNA3 | 2.669 | 17.08 |
| Standard DNA4 | 1.669 | 19.91 |
| Standard DNA5 | 0.669 | 23.15 |

A standard curve was created using the average of the actual concentrations of the 5 standard DNA stock solutions calculated via the actual concentrations of the stock solutions extrapolated from the dPCR results and the Cq values obtained from the standard DNA with 4 different concentrations, and the concentration of the 5th standard DNA was calculated. The linear regression equation (y: Cq; x: log(copy number/well)), R², PCR efficiency, Cq value of target DNA and copy number/well extrapolated therefrom, and average copy number/well detected by dPCR are listed in Table 5 below. As shown in Table 5, in the extrapolation results of the 5 standard DNA, the ratios of the copy number/well (A) calculated by the Cq values and the average copy number/well (B) detected by dPCR are all close to 1. Therefore, it may be known that the results of the copy number/well (A) calculated by the Cq values and the average copy number/well (B) detected by dPCR are quite close.

TABLE 5

| | | | | Target | Copy number/ | Average copy number/ | |
|---|---|---|---|---|---|---|---|
| Linear regression | | PCR | Target | DNA Cq | well (A) calculated | well (B) detected | |
| equation | R² | efficiency | DNA | value | by Cq value | by dPCR | A/B |
| y = −3.058x + 25.143 | 0.9994 | 112.33% | Standard | 10.09 | 8.37 × 10⁴ | 4.67 × 10⁴ | 1.79 |
| | | | DNA1 | | | | |
| y = −3.254x + 25.429 | 0.9984 | 102.91% | Standard | 13.90 | 3.49 × 10³ | 4.67 × 10³ | 0.75 |
| | | | DNA2 | | | | |
| y = −3.213x + 25.338 | 0.9982 | 104.76% | Standard | 17.08 | 3.72 × 10² | 4.67 × 10² | 0.80 |
| | | | DNA3 | | | | |
| y = −3.2314x + 25.488 | 0.9973 | 103.92% | Standard | 19.91 | 5.32 × 10¹ | 4.67 × 10¹ | 1.14 |
| | | | DNA4 | | | | |
| y = −3.264x + 25.589 | 0.9954 | 102.48% | Standard | 23.15 | 5.59 | 4.67 | 1.20 |
| | | | DNA5 | | | | |

### Sample Analysis Test

The expression of different mRNA in human cell line was analyzed using the test carrier PanelChip (manufactured by Quark Biosciences, Inc.) having a plurality of reaction wells. The plurality of reaction wells of the PanelChip were divided into 32 clusters, wherein each cluster was made up of 3×3 reaction wells, each cluster was provided with the primers of standard DNA or target DNA in advance, and each cluster on the PanelChip had three repetitions. The target DNA of 32 clusters are shown in Table 6. The average copy number of the standard DNA in each reaction well is shown in Table 7.

TABLE 6

| | Target DNA |
|---|---|
| 1 | Standard DNA1 |
| 2 | Standard DNA2 |
| 3 | Standard DNA3 |
| 4 | Standard DNA4 |
| 5 | Standard DNA5 |
| 6 | ALK |
| 7 | AR-V7 |
| 8 | β-actin |
| 9 | BRAF |
| 10 | BRCA1 |
| 11 | BRCA2 |
| 12 | CDK6 |
| 13 | CTNNB1 |
| 14 | EGFR |
| 15 | ERBB2 |
| 16 | ERBB3 |
| 17 | ERBB4 |
| 18 | HBAC100 |
| 19 | HER2 |
| 20 | HPRT |
| 21 | IDHI |
| 22 | KIT |
| 23 | KRAS |
| 24 | MET |
| 25 | PD-1 |
| 26 | PD-L1 |
| 27 | Buffer |
| 28 | PTEN |
| 29 | pUC57 (miRNA) |
| 30 | qPCR-control |
| 31 | RT-control |
| 32 | SMO |

TABLE 7

| | Average copy number in each well |
|---|---|
| Standard DNA1 | 2.33 × 10⁴ |
| Standard DNA2 | 2.33 × 10³ |
| Standard DNA3 | 2.33 × 10² |
| Standard DNA4 | 2.33 × 10¹ |
| Standard DNA5 | 2.33 |

### Test 1

A standard curve was created for the 5 standard DNA, and the equation is y=−3.3046x+26.104, wherein R² is 0.9974. The log(average copy number/well) and the average Cq values of the 5 standard DNA are listed in Table 8 below, and the PCR efficiency is 100.73%.

TABLE 8

| | log(average copy number/well) | Average Cq value |
|---|---|---|
| Standard DNA1 | 4.37 | 11.593 |
| Standard DNA2 | 3.37 | 15.239 |
| Standard DNA3 | 2.37 | 18.223 |
| Standard DNA4 | 1.37 | 21.205 |
| Standard DNA5 | 0.37 | 25.133 |

### Test 2

The 5 standard DNA were added into HBAC100 standard for testing and a standard curve was created for the 5 standard DNA. The equation is y=−3.2074x+25.482, and R² is 0.9968. The log(average copy number/well) and the average Cq values of the 5 standard DNA are listed in Table 9 below, and the PCR efficiency is 105.01%. The average Cq value, log(copy number/well), copy number/well, stock solution concentration (copy number/μl) (A) calculated, and stock solution concentration (copy number/μl) detected by dPCR of the HBAC100 standard are listed in Table 10 below. As shown in Table 10, in the extrapolation result of the HBAC100 standard, the ratios of the stock solution concentration (copy number/μl) (A) calculated and the stock solution concentration (copy number/μl) (B) detected by dPCR are all close to 1. Therefore, it may be known that the results of the stock solution concentration (copy number/μl) (A) calculated and the stock solution concentration (copy number/μl) (B) detected by dPCR are quite close, and even when mixed with other standards (HBAC100 standard), the performance of the absolute quantification method of the invention is not affected.

TABLE 9

| | log(copy number/well) | Average Cq value |
|---|---|---|
| Standard DNA1 | 4.37 | 11.14 |
| Standard DNA2 | 3.37 | 14.962 |
| Standard DNA3 | 2.37 | 18.028 |
| Standard DNA4 | 1.37 | 21.29 |
| Standard DNA5 | 0.37 | 24.013 |

TABLE 10

| | | | | Stock solution | |
|---|---|---|---|---|---|
| | | | Calculated | concentration | |
| | | | stock solution | detected by | |
| Average | log(copy | Copy | concentration | dPCR | |
| Cq | number/ | number/ | (copy number/ | (copy number/ | |
| value | well) | well | μl) (A) | μl) (B) | A/B |
| 17.74 | 2.41 | 2.57 × 10² | 1.72 × 10⁸ | 1.78 × 10⁸ | 0.97 |

### Test 3

The 5 standard DNA were added into a 10-fold dilution of A549 cell line cDNA for testing, and a standard curve was created for the 5 standard DNA. The equation is y=−3.3129x+25.669, and R² is 0.9974. The log(average copy number/well) and the average Cq values of the 5 standard DNA are listed in Table 11 below, and the PCR efficiency is 100.38%. The average Cq value, log(copy number/well), copy number/well (A) calculated, and copy number/well (B) of dPCR obtained for different target genes in the A549 cell line cDNA are listed in Table 12 below. As shown in Table 12, in the test result of different target genes in the A549 cell line cDNA, the ratios of the copy number/well (A) calculated and the copy number/well (B) of dPCR are all close to 1. Therefore, it may be known that the results of the copy number/well (A) calculated and the copy number/well (B) of dPCR are quite close. In the case of actually measuring a sample, the absolute quantification method of the invention shows the desired performance result when the 5 standard DNA were mixed with a human cell line for target gene measurement.

TABLE 11

| | log(average copy number/well) | Average Cq value |
|---|---|---|
| Standard DNA1 | 4.37 | 10.912 |
| Standard DNA2 | 3.37 | 14.902 |
| Standard DNA3 | 2.37 | 17.914 |
| Standard DNA4 | 1.37 | 20.923 |
| Standard DNA5 | 0.37 | 24.466 |

TABLE 12

| | | | | Stock solution | |
|---|---|---|---|---|---|
| | | | Calculated | concentration | |
| | | | stock solution | detected by | |
| | Average | log(copy | concentration | dPCR | |
| Target | Cq | number/ | (copy number/ | (copy number/ | |
| gene | value | well) | μl) (A) | μl) (B) | A/B |
| β-actin | 16.86 | 2.659 | 4.56 × 10² | 1.03 × 10³ | 0.44 |
| HPRT | 22.184 | 1.052 | 1.13 × 10¹ | 1.46 × 10¹ | 0.77 |
| IDHI | 20.449 | 1.576 | 3.76 × 10¹ | 5.07 × 10¹ | 0.74 |
| MET | 22.211 | 1.044 | 1.11 × 10¹ | 2.92 × 10¹ | 0.38 |
| PTEN | 22.344 | 1.004 | 1.01 × 10¹ | 1.73 × 10¹ | 0.58 |

Based on the above, the invention provides a method of multi-gene absolute quantification for PCR array, which is suitable for a test carrier having a plurality of reaction wells. Primers of different standard DNA or target DNA are respectively disposed in the reaction wells of the test carrier, wherein the plurality of standard DNA have different forward primers and reverse primers respectively, and the DNA sequences of the forward primers and reverse primers are different and sequence-specific. Therefore, interference between the plurality of standard DNA can be avoided. In addition, the primers for amplifying the plurality of standard DNA and the nucleic acid target to be tested have similar amplification efficiencies. As a result, the plurality of standard DNA and the nucleic acid sample are mixed and added into the reaction wells in the invention, and then a standard curve can be created according to the different copy numbers and Cq values of the plurality of standard DNA, so as to extrapolate the copy number of the nucleic acid target. Therefore, interference between the primers of the standard DNA and the sample to be tested can be effectively avoided, so it is not necessary to dispose the standard DNA and the sample to be tested in different reaction wells for testing. In this manner, the drawback of having to use additional wells to perform testing on standard DNA in the prior art can be alleviated, such that the experimental process is facilitated and the testing cost is reduced.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A method of a multi-gene absolute quantification for a PCR array, comprising:
respectively disposing primers for amplifying a plurality of standard DNA or nucleic acid target to be tested in a plurality of reaction wells of a test carrier; and
mixing and adding the plurality of standard DNA with known but different copy numbers and a nucleic acid sample into the plurality of reaction wells, wherein the nucleic acid sample contains at least one kind of the nucleic acid target, and a qPCR is performed on the plurality of standard DNA and the nucleic acid sample,
wherein a sequence-specific combination of a forward primer and a reverse primer is designed for each of the standard DNA, a same DNA template sequence is presented between regions corresponding to the forward primer and the reverse primer, and the primers for amplifying the plurality of standard DNA and the nucleic acid target to be tested have similar amplification efficiencies.

2. The method of the multi-gene absolute quantification for the PCR array of claim 1, further comprising making a standard curve according to the different copy numbers and Cq values of the plurality of standard DNA with a log(copy number) as a horizontal axis and the Cq value as a vertical axis, and then substituting a Cq value of the nucleic acid target into the standard curve to obtain a copy number of the nucleic acid target.

3. The method of the multi-gene absolute quantification for the PCR array of claim 1, wherein the amplification efficiencies comprise a Tm value.

4. The method of the multi-gene absolute quantification for the PCR array of claim 1, wherein the plurality of reaction wells of the test carrier are divided into a plurality of clusters, each of the clusters is formed by the plurality of reaction wells, and a same kind of primers for amplifying the standard DNA or the nucleic acid target are disposed in the plurality of reaction wells of each of the clusters.

5. The method of the multi-gene absolute quantification for the PCR array of claim 4, wherein the reaction wells of each of the clusters are in a 3×3 format.

6. The method of the multi-gene absolute quantification for the PCR array of claim 4, wherein each of the clusters is used to perform a qPCR on one of the plurality of standard DNA or a single kind of the nucleic acid target.
